(19) United States
(12) Patent Application Publication
(10) Pub. No.: US 2022/0211179 A1
(43) Pub. Date: Jul. 7, 2022

(54) Active Posture Management

(71) Applicant: Karthik Vasanth, Richardson, TX (US)

(72) Inventor: Keshav Vasanth, Richardson, TX (US)

(21) Appl. No.: 17/249,689

(22) Filed: Mar. 10, 2021

Related U.S. Application Data
(60) Provisional application No. 63/133,389, filed on Jan. 3, 2021.

(51) Int. Cl. A47C 7/46; G05B 6/02; G01V 8/10; G01S 15/04

(52) U.S. Cl. CPC A47C 7/467; G05B 6/02; A61B 5/4561; G01S 15/04; G01V 8/10

(57) ABSTRACT

An apparatus for managing posture may comprise a back support, a sensor unit coupled to the back support, a posture manipulator coupled to the back support, and a controller coupled to the sensor unit and to the posture manipulator. The controller may be configured to receive a feedback signal from the sensor unit indicative of posture, and to operate the posture manipulator based on the feedback signal. Some embodiments of the apparatus may be configured to compare the feedback signal to a posture model and operate the posture model based on a difference between the feedback signal and the posture model. The sensor unit may comprise one or more sensors for determining various biometric and biomechanical parameters.

### TECHNICAL FIELD

The invention set forth in the appended claims relates generally to active posture management. More particularly, but without limitation, the claimed subject matter relates to systems, apparatuses, and methods for actively managing posture while in a seated position.

### BACKGROUND

Back pain, and especially lower back pain, is a leading cause of lost productivity and disability. Back pain afflicts approximately 80% of the U.S. population at some point in time and the cost of treating back pain can be very significant.

Back pain can often be the result of sitting with incorrect posture for extended periods of time, such as sitting at a table or in front of a computer screen.

While the benefits of improved posture may be widely known, improvements to systems, apparatuses, and methods for managing posture can continue to benefit people by reducing or preventing back pain.

### BRIEF SUMMARY

New and useful systems, apparatuses, and methods for managing posture are set forth in the appended claims. Illustrative embodiments are also provided to enable a person skilled in the art to make and use the claimed subject matter.

For example, an illustrative embodiment may comprise a smart, connected sensor system for ergonomic posture detection, correction, and optimization. The sensor system may comprise pressure sensors placed to yield a pressure map of critical areas, and the pressure map may be used to derive posture information. In some examples, the posture information may be used to create a posture model. Other types of sensors may additionally, or alternatively, collect other biometric and/or biomechanical information.

In some embodiments, a system for managing posture may comprise a pad with sensors to assess posture. Additionally, or alternatively, the pad may comprise active devices to correct posture. Some embodiments of the pad may fit over a chair, for example, or may be manufactured with a chair. An application on a smart phone or other computer may be configured to access information from the sensors and control the active devices based on information from the sensors. Some embodiments may alert a person to modify their position. For example, a smart phone may be configured to use voice alerts to avoid sitting for too long or keeping legs in one position for too long.

Additionally, or alternatively, some embodiments may be configured to determine optimal head and neck posture. For example, some embodiments may comprise a head support having infrared sensors, which can be used to measure head placement, neck placement, or both. Ultrasound may also be used in some embodiments to detect posture and distance from a back support and/or a head support.

Active devices, such as inflatable cushions or bladders can be operated to reorient a person into a desired posture. For example, some embodiments may comprise or consist of a chair that can be reconfigured to accommodate different people and different postures, as well as reorient a person into a desired posture.

Some embodiments may additionally be configurable based on biometric input, such as gender, height, weight, and age, which can be used to determine an ideal posture model. The ideal posture model may additionally be configured based on activity or user preference. For example, an ideal posture model may be selected based on subjective comfort considerations rather than objective technical considerations.

More generally, an apparatus for managing posture may comprise a back support, a sensor unit coupled to the back support, a posture manipulator coupled to the back support, and a controller coupled to the sensor unit and to the posture manipulator. The controller may be configured to receive a feedback signal from the sensor unit indicative of posture, and to operate the posture manipulator based on the feedback signal. For example, some embodiments of the apparatus may be configured to compare the feedback signal to a posture model and operate the posture manipulator based on a difference between the feedback signal and the posture model. The sensor unit may comprise one or more sensors for determining various biometric and biomechanical parameters. For example, some embodiments of a sensor unit may comprise one or more of a pressure sensor, a proximity sensory, and a position sensor.

In some embodiments, a posture manipulator may comprise a bladder having a fluid disposed therein, and a controller can be configured to operate the posture manipulator by modifying pressure of the fluid in the bladder. Additionally, or alternatively, some embodiments of the posture manipulator may comprise a pressure plate and an actuator coupled to the pressure plate. A controller may be configured to operate the posture manipulator by moving the actuator.

Some embodiments of an apparatus for managing posture may comprise a back support, a sensor unit coupled to the back support, a posture manipulator, and a transceiver coupled to the sensor unit and to the posture manipulator. The transceiver can be configured to communicate signals between the sensor unit, the posture manipulator, and a controller. The sensor unit may comprise one or more sensors for determining biometric and biomechanical parameters, such as pressure sensors, proximity sensors, and position sensors. For example, ultrasonic or infrared sensors may be configured to measure proximity in some embodiments.

Some embodiments of a method for managing posture may comprise receiving a feedback signal from a sensor indicative of posture in a back support, comparing the feedback signal to a posture model, and operating a posture manipulator based on a difference between the feedback signal and the posture model. In some embodiments, the feedback signal may comprise one or more of a pressure signal, a proximity signal, and a position signal.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is a functional block diagram of an example embodiment of an apparatus for managing posture in accordance with this specification.

FIG. 2 is a schematic diagram of an example of the system of FIG. 1, illustrating additional details that may be associated with some embodiments.

FIG. 3 is a front view of the example system of FIG. 2;

FIG. 4 is a section view of the example system of FIG. 3;

FIG. 5 is a schematic diagram of another example of the system of FIG. 1, illustrating additional details that may be associated with some embodiments.

FIG. 6 is a schematic diagram of another example of the system of FIG. 1, illustrating additional details that may be associated with some embodiments.

FIG. 7 is a flowchart of an example process for actively managing posture with the system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a person in a seated position. However, as should be recognized by those skilled in the art, this frame of reference is merely a descriptive expedient rather than a strict prescription.

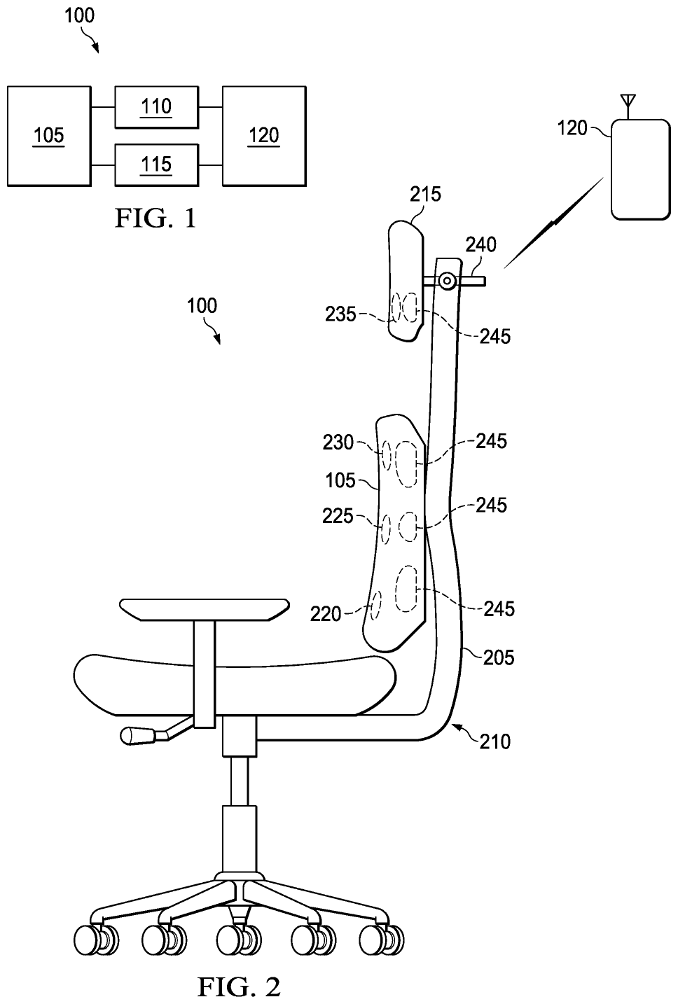

FIG. 1 is a functional block diagram of an example of a system 100 for actively managing posture. In the example of FIG. 1, the system 100 comprises a back support 105, a sensor unit 110 coupled to the back support 105, a posture manipulator 115 coupled to the back support 105, and a controller 120 coupled to the sensor unit 110 and to the posture manipulator 115.

In general, components of the system 100 may be coupled directly or indirectly. For example, the back support 105 may be directly coupled to the sensor unit 110 and may be indirectly coupled to the controller 120 through the sensor unit 110. Coupling may include fluid, mechanical, thermal, electrical, or chemical coupling (such as a chemical bond), or some combination of coupling in some contexts. For example, the posture manipulator 115 may be mechanically coupled to the back support 105 and may be electrically coupled to the controller 120. In some embodiments, components may also be coupled by virtue of physical proximity, being integral to a single structure, or being formed from the same piece of material.

Additionally, some components of the system 100 may be configured to exchange signals over a communication medium, which may include any physical, optical, electromagnetic, or other medium through which data can be transmitted. For example, the controller 120 may be configured to exchange signals with the sensor unit 110 and the posture manipulator 115 over a suitable electromagnetic medium. In some embodiments, signals may be exchanged using standard protocols, such as the BLUETOOTH or WI-FI standards.

The sensor unit 110 may comprise one or more sensors, which are generally known in the art as any apparatus operable to detect or measure a physical phenomenon or property, and generally provide a signal indicative of the phenomenon or property that is detected or measured. For example, the sensor unit 110 may be configured to measure one or more operating parameters of the system 100. In some embodiments, for example, the sensor unit 110 may comprise a pressure sensor, such as a transducer configured to measure pressure applied to some portion of the back support 105. Other suitable sensors may include proximity sensors, position sensors, heart-rate sensors, and temperature sensors. For example, ultrasonic sensors or infrared sensors may be configured to determine proximity and position in some embodiments. The sensor unit 110 may convert the measurement to a signal indicative of the pressure measured. In some embodiments, a piezo-resistive strain gauge may be a suitable pressure sensor. The sensor unit 110 may optionally measure other operating parameters, such as a voltage or current applied to the posture manipulator 115. Preferably, the signals from the sensor unit 110 are suitable as an input signal to the controller 120, but some signal conditioning may be appropriate in some embodiments. For example, the signal may need to be filtered or amplified before it can be processed by the controller 120. Typically, the signal is an electrical signal, but may be represented in other forms, such as an optical signal.

In some examples, the controller 120 may be implemented with a variety of processing hardware, software, or both. In more particular examples, the controller 120 may comprise or consist essentially of a microprocessor programmed to operate one or more components of the system 100, such as the posture manipulator 115. In some embodiments, for example, the controller 120 may be a microcontroller, which generally comprises an integrated circuit containing a processor core and a memory programmed to directly or indirectly control one or more operating parameters of the system 100. The controller 120 may also be configured to receive one or more input signals, such as a feedback signal from the sensor unit 110, and may be programmed to modify one or more operating parameters based on the input signals.

Other suitable processing hardware may include any machine or article that is capable of accepting, performing logic operations on, storing, or displaying data, which may include one or more microprocessors and memory. Logic for the controller 120 may be encoded or otherwise stored in persistent memory or tangible media, such as embedded logic provided in an application-specific integrated circuit or digital signal processor instructions. Memory may encompass and be distributed across a plurality of media. In some examples, at least some part of logic for the controller 120 may be implemented with fixed logic or programmable logic in a programmable processor, a field programmable gate array, an erasable programmable read-only memory, an electrically erasable programmable read-only memory, or an application-specific integrated circuit that includes digital logic, software, code, electronic instructions, or any suitable combination thereof. The logic may comprise a plurality of smaller programming units, including without limitation subroutines, modules, functions, methods, and procedures. Thus, the functions of the controller 120 may be distributed among a variety of hardware and software.

In more particular embodiments, at least some of the logic and functions of the controller 120 may be implemented as a software application, which can receive data from the sensor unit 110 to evaluate the posture of a person in contact with the back support 105 and operate the posture manipulator 115 to actively manage the posture.

The controller 120 may be configured to receive a feedback signal from the sensor unit 110, which may be indicative of posture. For example, if a person is resting their back against the back support 105, the sensor unit 110 may measure pressure applied to various areas of the back support 105 and generate a feedback signal representative of the pressure in each area, which can be transmitted to the controller 120. The controller 120 may access a posture model that represents an ideal posture, and then compare the feedback signal to the posture model. For example, in some embodiments a posture model may be represented as a two-dimensional array or map of pressure or position data. In some embodiments, if the feedback signal indicates a deviation from the posture model, the controller 120 may additionally compare the deviation to a threshold value to determine the probability of the deviation causing back strain. In some embodiments, the controller 120 may prompt a person to enter personal data, such as height, weight, gender, and age, and the controller 120 can select from more than one posture model based on the personal data.

In more particular examples, the controller 120 may additionally be configured to alert the person and provide recommendations for correcting the posture. For example, the controller 120 may provide a textual alert or a graphical interface that displays representation of the feedback signal and the posture model. Additionally, or alternatively, the controller 120 may provide audible or haptic alerts. In some embodiments, for example, a haptic alert may indicate a location in the back support 105 where back position or pressure should be adjusted.

The controller 120 can operate the posture manipulator 115 based on the feedback signal from the sensor unit 110. In some embodiments, for example, the controller 120 may operate the posture manipulator 115 if the difference between the feedback signal and the posture model exceeds a threshold value.

FIG. 2 is a schematic diagram of an example of the system 100, illustrating additional details that may be associated with some embodiments. In FIG. 2, for example, the back support 105 may be coupled to a back frame 205 of a chair 210. In other examples, the back support 105 may be configured to cover the back frame 205 or a cushion or pad coupled to the back frame 205. Some embodiments of the system 100 may additionally include a head support 215, as illustrated in FIG. 2. The controller 120 may be implemented in a mobile phone or other handheld device configured to communicate wirelessly with the sensor unit 110.

As illustrated in the example of FIG. 2, the sensor unit 110 may comprise a first sensor 220, a second sensor 225, and a third sensor 230, each of which may be integral with the back support 105. For example, the first sensor 220 may be a pressure and/or proximity sensor that is disposed near a base of the back support 105 to measure the degree of contact with the lower back of a person seated in the chair 210. The second sensor 225 may also be a pressure and/or proximity sensor, which can be centrally disposed within the back support 105 to measure the distance or degree of contact with the mid-back of a person seated in the chair 210. The third sensor 230 may be disposed near a top of the back support 105 to measure the distance or degree of contact with the upper back of a person seated in the chair 210. In general, any number of sensors may be distributed throughout the back support 105. Additionally, or alternatively, some embodiments may include a means for focusing pressure applied across a relatively large surface area onto one or more of the sensors. For example, some embodiments of the back support 105 may include a substantially rigid support layer, such as a Styrofoam block, which can focus weight from a portion of a person's back onto a sensor.

The sensor unit 110 may comprise a fourth sensor 235 coupled to the head support 215, which can be configured to measure the distance or degree of contact with the back of the head of a person seated in the chair 210. In the example of FIG. 2, the head support 215 is mounted on a rod 240, which is coupled to the back frame 205. The position of the head support 215 may be adjusted by the controller 120 based on the feedback signal, personal data, or both, or may be adjusted manually with a control knob.

As illustrated in the example of FIG. 2, some embodiments of the posture manipulator 115 may comprise one or more bladders 245, which may be distributed throughout the back support 105. In the example of FIG. 2, the bladders 245 are disposed posterior to the sensor unit 110. The relative position of the bladders 245 and the sensor unit 110 may vary in other embodiments. For example, the bladders 245 may be anterior or aligned with sensors in some embodiments.

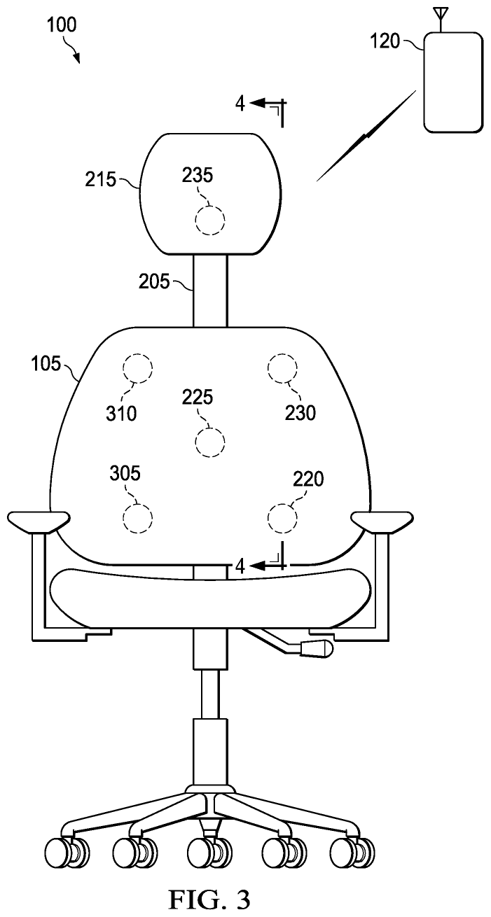

FIG. 3 is a front view of the system 100 of FIG. 2, illustrating additional details that may be associated with some embodiments. As illustrated in the example of FIG. 3, some embodiments of the sensor unit 110 may have additional sensors distributed across the back support 105 to form an array or grid of sensors. In FIG. 3, for example, the sensor unit 110 comprises a sensor 305 and a sensor 310.

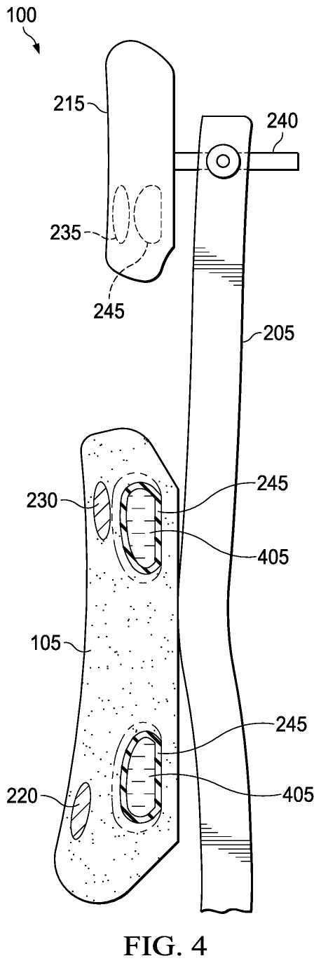

FIG. 4 is a section view of the system 100 taken along line 4-4 of FIG. 3. As shown in FIG. 4, a fluid 405 may be disposed within each of the bladders 245, and the controller 120 may be configured to modify the pressure of the fluid 405 to operate the posture manipulator 115. For example, the system 100 may have one or more pumps (not shown) that can be operated by the controller 120 to increase or decrease pressure in the bladders 245 based on the feedback signal from the sensor unit 110. Increasing the pressure can cause the bladders 245 to expand, as illustrated by the phantom lines in FIG. 4, which can modify the pressure or support in various areas of person's back that may be in contact with the back support 105.

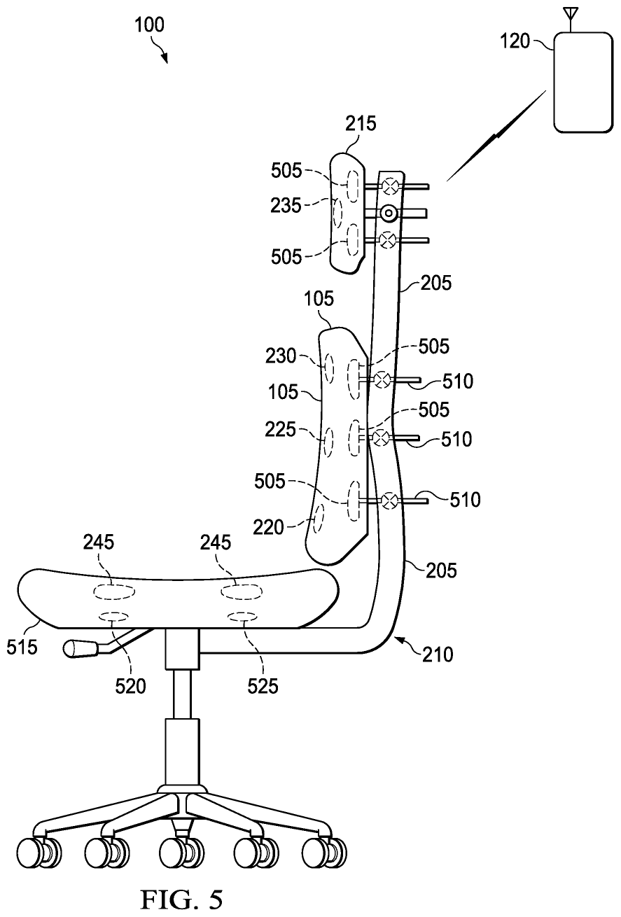

FIG. 5 is a schematic diagram of another example of the system 100, illustrating additional details that may be associated with some embodiments. In FIG. 5, the back support 105 is coupled to the back frame 205 of the chair 210. The system 100 of FIG. 5 also includes the head support 215. The controller 120 may be implemented in a mobile phone or other handheld device configured to communicate wirelessly with the sensor unit 110.

In the example of FIG. 5, the posture manipulator 115 may comprise one or more pressure plates 505. In FIG. 5, the pressure plates 505 are coupled to linear actuators 510, which may be mounted on the back frame 205. In some embodiments, the linear actuators 510 may comprise motors, solenoids, or pneumatic pistons, for example. The controller 120 can be configured to operate the posture manipulator 115 by moving one or more of the linear actuators 510 based on the feedback signal from one or more of the first sensor 220, the second sensor 225, the third sensor 230, or the fourth sensor 235. Moving the linear actuators 510, in turn, can move the pressure plates 505. For example, the pressure plates 505 can be extended to increase pressure or support on a person's back that is in contact with the back support 105, or can be retracted to decrease pressure or support on the back.

Additionally, some embodiments of the system 100 may comprise a seat support 515, as illustrated in the example of FIG. 5. The seat support 515 may also be coupled to the sensor unit 110. In FIG. 5, for example, the sensor unit 110 may comprise a fifth sensor 520 and a sixth sensor 525, which may be integral to or otherwise coupled to the seat support 515. The posture manipulator 115 may also be coupled to the seat support 515 in some embodiments. For example, bladders 245 may be integral to the seat support 515, as illustrated in FIG. 5. In other examples, the posture manipulator 115 may comprise pressure plates and linear actuators coupled to the seat support 515, similar or analogous to the examples in the back support 105 of FIG. 2.

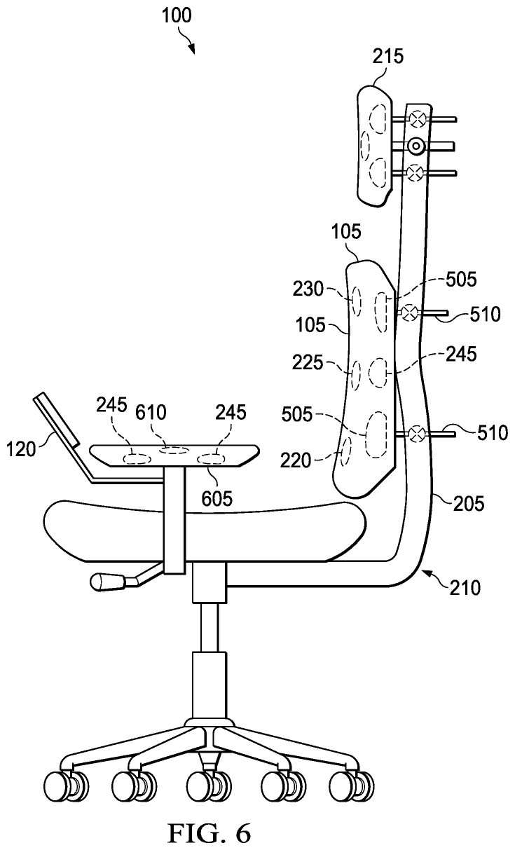

FIG. 6 is a schematic diagram of another example of the system 100, illustrating additional details that may be associated with some embodiments. In the example of FIG. 6, the back support 105 is coupled to the back frame 205 of the chair 210. The system 100 of FIG. 6 also includes the head support 215. FIG. 6 illustrates an example of the controller 120 implemented as a tablet computer or other similar apparatus mounted to an arm support 605. The controller 120 of FIG. 6 may be configured to communicate wirelessly with the sensor unit 110 or may be wired to the sensor unit 110.

In the example of FIG. 6, the first sensor 220, the second sensor 225, and the third sensor 230 are each integral with the back support 105. Some embodiments of the system 100 may additionally, or alternatively, comprise a seventh sensor 610 coupled to the arm support 605.

The posture manipulator of FIG. 6 comprises one of the bladders 245 and two of the pressure plates 505 coupled to linear actuators 510. The posture manipulator 115 may also be coupled to the arm support 605 in some embodiments. For example, the arm support 605 of FIG. 6 is coupled to two of the bladders 245.

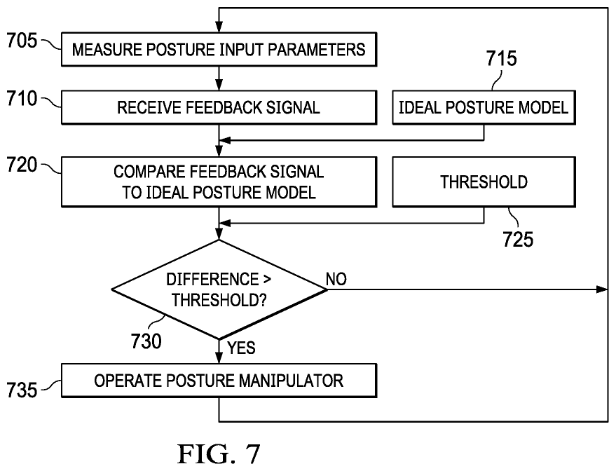

FIG. 7 is a flowchart of an example process for actively managing posture with the system 100, illustrating additional details that may be associated with some embodiments. In some embodiments, some or all of the steps of FIG. 7 may be implemented with or in the sensor unit 110, the posture manipulator 115, and the controller 120. For example, at step 705 in the process of FIG. 7, certain input parameters indicative of posture may be measured with the sensor unit 110. In some embodiments, the posture input parameters may include pressure applied to the back support 105, for example. The sensor unit 110 may also determine other biometric and biomechanical input parameters, such as proximity, position, heart rate, and/or temperature. At step 710, the controller 120 may receive feedback signals from the sensor unit 110, which can be indicative of the input parameters measured by the sensor unit 110. The controller 120 may compare the feedback signal to an ideal posture model 715 at step 720. For example, in some embodiments, the controller 120 may convert the feedback signals to an active posture model, which can be compared to the ideal posture model 715. In some embodiments, the active posture model, the ideal posture model 715, or both may be represented as a pressure map. For example, an active posture model may be represented as a two-dimensional array of pressure data corresponding to an array of sensors in some embodiments of the sensor unit 110. If the difference between the feedback signal and the ideal posture model 715 exceeds a configurable, predetermined threshold 725 at step 730, the controller 120 may operate the posture manipulator 115 at step 735 to decrease the difference between the feedback signal and the ideal posture model 715. For example, if the feedback signal indicates the difference in one area exceeds the predetermined threshold 725, the controller 120 may operate the posture manipulator 115 nearest to the area to counteract the difference. If there is no difference, or if the difference does not exceed the threshold, the sensor unit 110 continues to measure the input parameters at step 705.

The systems, apparatuses, and methods described herein may provide significant advantages. For example, some embodiments may be particularly advantageous for actively managing posture while seated. Actively managing posture can substantially reduce or eliminate back pain, which can be a significant contributor to healthcare costs and lost productivity.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations, the back support 105, the sensor unit 110, the posture manipulator 115, and the controller 120 may each be separated from one another or combined in various ways for manufacture or sale.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for managing posture, the apparatus comprising:
a back support;
a sensor unit coupled to the back support;
a posture manipulator coupled to the back support; and
a controller coupled to the sensor unit and to the posture manipulator, the controller configured to:
receive a feedback signal from the sensor unit indicative of posture, and
operate the posture manipulator based on the feedback signal.

2. The apparatus of claim 1, wherein the controller is configured to:
compare the feedback signal to a posture model; and
operate the posture manipulator based on a difference between the feedback signal and the posture model.

3. The apparatus of claim 1, wherein the sensor unit comprises one or more of a pressure sensor, a proximity sensor, and a position sensor.

4. The apparatus of claim 1, wherein the sensor unit comprises ultrasonic or infrared sensors configured to measure proximity.

5. The apparatus of claim 1, wherein the posture manipulator comprises a bladder having a fluid disposed within the bladder.

6. The apparatus of claim 5, wherein the controller is configured to operate the posture manipulator by modifying a pressure of the fluid disposed within the bladder.

7. The apparatus of claim 1, wherein the posture manipulator comprises a pressure plate and a linear actuator coupled to the pressure plate.

8. The apparatus of claim ₇, wherein the controller is configured to operate the posture manipulator by moving the linear actuator.

9. The apparatus of claim 1, further comprising a seat support coupled to the sensor unit and to the posture manipulator.

10. An apparatus for managing posture, the apparatus comprising:
a back support;
a sensor unit coupled to the back support;
a posture manipulator coupled to the back support; and
a transceiver coupled to the sensor unit and to the posture manipulator, the transceiver configured to communicate signals between the sensor unit, the posture manipulator, and a controller.

11. The apparatus of claim 10, wherein the sensor unit comprises one or more of a pressure sensor, a proximity sensor, and a position sensor.

12. The apparatus of claim 10, wherein the sensor unit comprises ultrasonic or infrared sensors configured to measure proximity.

13. The apparatus of claim 10, wherein the posture manipulator comprises a bladder having a fluid disposed within the bladder.

14. The apparatus of claim 10, wherein the posture manipulator comprises a pressure plate and a linear actuator coupled to the pressure plate.

15. A method for managing posture, the method comprising:
receiving a feedback signal from a sensor indicative of posture in a back support;
comparing the feedback signal to a posture model; and
operating a posture manipulator based on a difference between the feedback signal and the posture model.

16. The method of claim 15, wherein the feedback signal comprises one or more of a pressure signal, a proximity signal, and a position signal.

17. The method of claim 15, wherein the feedback signal comprises a proximity signal from an ultrasonic or infrared sensor.

18. The method of any of claim 15, wherein:
the posture manipulator comprises a bladder having a fluid disposed within the bladder; and
operating the posture manipulator comprises modifying a pressure of the fluid disposed within the bladder.

19. The method of claim 15, wherein:
the posture manipulator comprises a pressure plate and a linear actuator coupled to the pressure plate; and
operating the posture manipulator comprises moving the linear actuator.

20. (canceled)
